(19) United States
(12) Patent Application Publication
(10) Pub. No.: US 2009/0131801 A1
(43) Pub. Date: May 21, 2009

(54) SYSTEMS AND PROCESSES FOR OPTICAL IMAGING OF LUMINAL ANATOMIC STRUCTURES

(75) Inventors: Melissa J. Suter, Boston, MA (US); Guillermo J. Tearney, Cambridge, MA (US); Brett Eugene Bouma, Quincy, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/250,359

(22) Filed: Oct. 13, 2008

Related U.S. Application Data
(60) Provisional application No. 60/979,748, filed on Oct. 12, 2007.

(51) Int. Cl. A61B 6/00

(52) U.S. Cl. 600/478

(57) ABSTRACT

Exemplary embodiments of an apparatus for obtaining data for at least one portion within at least one luminal or hollow sample can be provided. For example, the exemplary apparatus can include a first optical arrangement configured to transceive at least one electromagnetic radiation to and from the portion. A second arrangement may be provided that can at least partially enclose the first arrangement. At least one third arrangement may be provided which is configured to be actuated so as to expand, at least in part, beyond a periphery of the second arrangement. Such exemplary third arrangement can be structured to facilitate a fluid flow and/or a gas flow therethrough. Further, a fourth arrangement may be provided which can be structured to (i) actuate a particular number of the third arrangement and/or (ii) adjust a distance between at least two outer portions of the third arrangement. According to one exemplary embodiment, the third arrangement can be a plurality of the third arrangements.

### CROSS-REFERENCE TO RELATED APPLICATION(S)

The present invention relates to U.S. provisional Application No. 60/979,748 filed Oct. 12, 2007, the entire disclosure of which is incorporated herein by reference.

### FIELD OF THE INVENTION

The present invention relates generally to systems and processes for optical imaging of variable diameter lumens or hollow organs and, more particularly to, e.g., exemplary embodiments of apparatus and processes for optical imaging of pulmonary airways.

### BACKGROUND INFORMATION

Lung cancer is the leading cause of cancer-related mortality in the western industrialized nations with a current 5-year survival rate under about 15%. (See Jemal A, Siegel R, Ward E, Murray T, Xu J, Thun M J. Cancer Statistics, 2007, CA: A Cancer Journal for Clinicians 2007; 57:43-66).

In the United States alone, lung cancer has been responsible for about 29% of all cancer-related deaths, approximately 160,000 deaths annually, more than breast, colorectal and prostate cancer combined. (See Jemal A, Siegel R, Ward E, Murray T, Xu J, Thun M J. Cancer Statistics, 2007, CA: A Cancer Journal for Clinicians 2007; 57:43-66; and Society AC, Cancer Facts & Figures 2007, American Cancer Society. Atlanta, 2007). Squamous cell carcinoma (SCC), or epidermoid carcinoma, accounts for 30% of all lung cancers (see Travis W D, Travis L B, S.S. D. Lung Cancer. Cancer 1995; 75:191-202) and in addition is the most lethal. The evolution of SCC occurs over many years in a step-wise progression, and generally presents in the main, lobar or segmental bronchi. (See id.) As smoking is the primary cause of SCC, lesions may develop multifocally, termed field carcinogenesis. (See Kerr K M, Pulmonary preinvasive neplasia, Journal of Clinical Pathology 2001; 54:257-271).

The initial stages may be characterized by a loss of the ciliated columnar epithelium, basal cell hyperplasia, and the development of cuboidal epithelium without cilia. (See id.) Disease progression generally continues with a development of squamous metaplasia, followed by various stages of dysplasia, carcinoma in situ, and finally invasive cancer. (See id.) In the early stages of disease development, the thickness of the lesions may be only a few cell layers deep (e.g., about 0.2-1 mm—see Hirsch F R, Franklin W A, Gazdar A F, Bunn P A. Early detection of lung cancer: clinical perspectives of recent advances in biology and radiology. Clinical Cancer Research 2001; 7:5-22) and may not be readily apparent with a conventional bronchoscopy (see Feller-Kopman D, Lunn W, Ernst A. Autofluorescence bronchoscopy and endobronchial ultrasound: a practical review, Annals of Thoracic Surgery 2005; 80:2395-2401), thus making the detection and diagnosis challenging.

Although significant efforts in the development of successful screening paradigms for the detection of lung cancer have been made, to date there is likely still no widely accepted and validated approach. Computed tomography (CT) and x-ray imaging typically does not detect early SCC as the lesions are generally radiographically occult. CT can predominately detect peripheral adenocarcinoma of the lung. The prevalence and high mortality rate associated with lung SCC and the lack of any widely accepted screening and surveillance tools can highlight the likely need for new imaging paradigms that will ultimately lead to a reduction in patient mortality.

### Optical Coherence Tomography

Optical coherence tomography (OCT) is a non-contact optical imaging modality that affords tomographic images of tissue in resolutions comparable with architectural histology (e.g., approximately <10 μm). One of the concepts of OCT is similar to that of ultrasound where measuring the delay of the source, as it is reflected off subsurface structures in biological tissues, generates depth information. Unlike ultrasound, however, a broadband light source can be used in OCT and, due to the high speed of light propagation in tissue, optical reflectance may be measured using low coherence interferometry. The broadband source can be separated into two arms, a reference arm and a sample arm. When the optical path length of the light traveled by each arm is identical the combined light from each channel forms and interference pattern. Thus, to construct a single depth profile, the reference arm reflector can be translated effectively changing the optical length of the reference arm and hence the penetration depth of the measured signal in the tissue. Three-dimensional images may be subsequently comprised of two-dimensional arrays of individual depth profiles. OCT can be advantageous in that it is a non-contact imaging technology that relies on endogenous contrast and may not require a transducing medium.

Certain preliminary ex vivo studies have been conducted regarding the use of optical coherence tomography (OCT) in the diagnosis of bronchial pathology. (See Yang Y, Whiteman S C, van Pittius D G, He Y, Wang R K, Spiteri M A, Use of optical coherence tomography in delineating airways microstructure: comparison of OCT images to histpathological sections, Physics in Medicine and Biology 2004; 49:1247-1255; Ikeda N, Hayashi A, Iwasaki K, Tsuboi M, Usuda J, Kato H, Comprehensive diagnostic bronchoscopy of central type early stage lung cancer, Lung Cancer 2007; 56:295-302; Tsuboi M, Hayashi A, Ikeda N, Honda H, Kato Y, Ichinose S, et al., Optical coherence tomography in the diagnosis of bronchial lesions, Lung Cancer 2005; 49:387-394; and Whiteman S C, Yang Y, van Pittius D G, Stephens M, Parmer J, Spiteri M A, Optical coherence tomography: real-time imaging of bronchial airways microstructure and detection of inflammatory/neoplastic morphologic changes, Clinical Cancer Research 2006; 12:813-818). These studies have demonstrated that OCT can indeed be used to visualize and evaluate the pulmonary tissue. However, such studies have generally been limited to small proof of concept trials with no definitive diagnostic criteria being developed. In addition, endoscopic OCT has also been used to interrogate the bronchial mucosa in a limited in vivo human proof of principle study. (See Tsuboi M, Hayashi A, Ikeda N, Honda H, Kato Y, Ichinose S, et al., Optical coherence tomography in the diagnosis of bronchial lesions, Lung Cancer 2005; 49:387-394).

Since SCC and its precursors can often be multifocal, and may arise anywhere within the major airways, a diagnostic tool, system and/or method for evaluating this disease should be able to investigate long bronchial segments during a clinically viable procedure time (e.g., about 1-5 minutes). While OCT has shown some promise for imaging pulmonary airways, its relatively slow speed can prohibit the screening of sufficiently large areas to be clinically useful. Further, a second-generation OCT technology—frequency domain imaging (OFDI)—has been developed. (See Yun S H, Teamey G J, de Boer J F, Iftimia N, Bouma B E, High-speed optical frequency-domain imaging. Optics Express 2003; 11:2953-2963). One of the advantages of OFDI is that this technique/procedure can provide images at rates that may be 100× faster than conventional OCT. Therefore, OFDI can be utilized to screen the bronchial tree in a manner that can be compatible with the temporal requirements of the bronchoscopy procedure. Volumetric imaging of the upper airways can solve certain dilemmas associated with screening and managing patients with SCC.

Systems and processes for detecting and diagnosing squamous cell carcinoma in the pulmonary airways may be needed to detect and treat precancerous lesions prior to such lesions progressing to malignant invasive cancers. Early detection through OCT and consequent treatment can lead to a consequent reduction in the mortality associated with the disease. OCT imaging of the pulmonary airways is an emerging field. Imaging the bronchial mucosa with this new technology has been demonstrated; however, to date, the full potential may not have been reached.

Indeed, there may be a need to overcome at least some of the deficiencies described above.

### OBJECTS AND SUMMARY OF EXEMPLARY EMBODIMENTS OF THE INVENTION

One of the objectives of the exemplary embodiments of the present invention is to overcome certain deficiencies and shortcomings of the conventional apparatus, and provide exemplary embodiments of apparatus and processes for optical imaging of pulmonary airways.

For example, exemplary embodiments of an apparatus for obtaining data for at least one portion within at least one luminal or hollow sample can be provided. For example, the exemplary apparatus can include a first optical arrangement configured to transceive at least one electromagnetic radiation to and from the portion. A second arrangement may be provided that can at least partially enclose the first arrangement. At least one third arrangement may be provided which is configured to be actuated so as to expand, at least in part, beyond a periphery of the second arrangement. Such exemplary third arrangement can be structured to facilitate a fluid flow and/or a gas flow therethrough. Further, a fourth arrangement may be provided which can be structured to (i) actuate a particular number of the third arrangement and/or (ii) adjust a distance between at least two outer portions of the third arrangement. According to one exemplary embodiment, the third arrangement can be a plurality of the third arrangements.

According to one exemplary variant, the third arrangement can be a wire arrangement and/or a plastic arrangement. Such wire arrangement may have at least one wire strand and/or a cage. Further, the third arrangement can include a balloon arrangement. Further, the third arrangement can have an approximately circular or elliptical outer periphery—e.g., a circumference of the third arrangement may be adjustable by the fourth arrangement. In addition, the fourth arrangement can actuates the particular number of the third arrangements. The third arrangements may be spaced apart from one another by at least one predetermined distance. The predetermined distance can be provided such that upon a completed collapse of each of the third arrangements, outer portions of the each of the third arrangements may be prevented from substantially overlapping one another. The third arrangements can be configured to be actuated to expand so as to be associated with a plurality of portions within the at least one luminal and/or hollow sample.

In still another exemplary embodiment of the present invention, the third arrangement can be statically connected to the second arrangement, and the third arrangement translates over at least one portion thereof. The third arrangement can adjust the distance by translating itself and/or the fourth arrangement with respect to one another. Further, in at least a partially expanded state, the third arrangement can have an approximate shape of a cone. The portion can be within an airway of a patient, and the third arrangement may be structured to be insertable into the airway.

According to a further exemplary embodiment of the present invention, the distance can be a radius of an outer periphery of the at least one third arrangement. A fifth arrangement can be provided that substantially surrounds the fourth arrangement. For example, the fifth arrangement can be an endoscope, a laparascope, a bronchoscope, a cystoscope and/or a guide catheter.

20. The apparatus according to claim 16, wherein the third arrangements are configured to be actuated to expand so as to be associated with a plurality of portions within the at least one luminal or hollow sample.

Other features and advantages of the present invention will become apparent upon reading the following detailed description of embodiments of the invention, when taken in conjunction with the appended claims.

### BRIEF DESCRIPTION OF THE DRAWINGS

Further objects, features and advantages of the present invention will become apparent from the following detailed description taken in conjunction with the accompanying figures showing illustrative embodiments of the present invention, in which:

FIG. 1 is a schematic diagram of an exemplary embodiment of an OFDI apparatus according to the present invention;

FIG. 2A is a schematic diagram of an exemplary embodiment of an OFDI probe configuration with a single balloon arrangement according to the present invention;

FIG. 2B is a schematic diagram of the exemplary embodiment of the OFDI probe of FIG. 2A in which where the imaging core is located adjacent to a lumen wall;

FIG. 2C is a schematic diagram of the exemplary OFDI probe configuration shown in FIG. 2A, in which the optical imaging core is centered within the lumen by a balloon arrangement;

FIG. 3A is an exemplary cross-sectional view of exemplary image data obtained using the exemplary embodiment of the OFDI probe configuration associated a single balloon arrangement according to the present invention;

FIG. 3B is a volume rendering image of the exemplary OFDI image data obtained using the OFDI probe configuration with the single balloon arrangement shown in FIG. 3A;

FIG. 3C is another volume rendering image of the OFDI image data obtained using the OFDI probe configuration with the single balloon arrangement shown in FIG. 3A;

FIG. 4A is a side view of a diagram of an exemplary embodiment of the OFDI probe configuration having multiple balloon arrangements with varying number and decreasing diameter properties to accommodate a lumen of decreasing diameter according to the present invention;

FIG. 4B is a side view of a diagram of another exemplary embodiment of the OFDI probe configuration having multiple balloon arrangements with varying number and diameter properties according to the present invention;

FIG. 4C is a side view of a diagram of a further exemplary embodiment of the OFDI probe configuration having two balloon arrangements with increasing diameter properties according to the present invention;

FIG. 5 is a side view of a diagram of yet another exemplary embodiment of the OFDI probe configuration having multiple wire cage arrangements with varying number and diameter properties according to the present invention; and

FIG. 6 is a side view of a diagram of an exemplary embodiment of the OFDI probe configuration having multiple umbrella-like wire arrangements with varying number and diameter properties according to the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

Herein, a detailed description of the principles of optical frequency domain imaging (OFDI) is provided, including preliminary results of comprehensive OFDI screening in an ex vivo swine airway.

### Imaging Technology

Optical Frequency Domain Imaging Optical frequency domain imaging (OFDI) can be a high speed second generation OCT imaging technology. (See, e.g., Yun S H, Tearney G J, de Boer J F, Iftimia N, Bouma B E, High-speed optical frequency-domain imaging. Optics Express 2003; 11:2953-2963). In traditional time domain OCT, a broadband light source can be used to illuminate both a reference and a sample arm. When the back-scattered light from the two arms has traveled the same optical distance, an interference pattern is formed which can be detected by a receiver. Individual depth profiles, or a-lines can then be obtained by mechanically translating the reference arm through the desired imaging depth range. Unlike OCT, OFDI utilizes a rapidly tuned wavelength swept laser source. (See, e.g., Yun S H, Tearney G J, de Boer J F, Iftimia N, Bouma B E, High-speed optical frequency-domain imaging. Optics Express 2003; 11:2953-2963; Brinkmeyer E, Ulrich R, High-resolution OCDR in dispersive waveguide, Electronic Letters 1990; 26:413-4; Chinn S R, E. S, Fujimoto J G, Optical coherence tomography using a frequency-tunable optical source, Optics Letters 1997; 22:340-2; Golubovic B, Bouma B E, Tearney G J, Fujimoto J G, Optical frequency-domain reflectometry using rapid wavelength tuning of a Cr4+:forsterite laser, Optics Letters 1997; 22:1704-6; Lexer F, Hitzenberger C K, Fercher A F, Kulhavy M, Wavelength-tuning interferometry of intraocular distances, Applied Optics 1997; 36:6548-53; and Yun S H, Boudoux C, Tearney G J, Bouma B E, High-speed wavelength-swept semiconductor laser with a polygon-scanner-based wavelength filter, Optics Letters 2003; 28:1981-3).

As differing wavelengths may penetrate the tissue to different depths an entire depth profile can be obtained simultaneously during a single sweep of the source while the reference arm remains stationary. A detection of the spectrally resolved interference between the sample and the stationary reference arm can then generate the depth profile. The interference signal may be detected by a set of balanced receivers, and the depth profile can be obtained by determining the Fourier transform. Due to the elimination of the mechanical translation of the reference arm, significantly higher OFDI imaging speeds may be attainable. In addition, the sensitivity of OFDI can be considerably higher than that of OCT due to the Fourier integration in the processing of the OFDI signal. (See, e.g., Yun S H, Tearney G J, de Boer J F, Iftimia N, Bouma B E, High-speed optical frequency-domain imaging. Optics Express 2003; 11:2953-2963). As the signal to noise ratio in OCT and OFDI imaging systems is proportional to the image power reflected from the sample and the image resolution, and inversely proportional to the acquisition speed and depth range, it is possible to image samples and/or portions thereof at significantly higher image acquisition speeds, compared to conventional OCT, without sacrificing image quality.

For example, a-line rates of up to about 64 kHz can be achievable with the exemplary OFDI procedures and systems. One exemplary embodiment of the OFDI system is configured to acquire, process and display image data at a sustained a-line rate of, e.g., about 52 kHz, corresponding to an imaging speed of, e.g., about >25 frames/sec (e.g., frame size: 1536×2048). The wavelength swept source for this exemplary system can be centered at about 1320 nm and may have a free spectral range (tuning range) of about 111 nm. This corresponds to an image ranging depth of approximately 4 mm and an axial resolution of about 5 microns in tissue (e.g., about n=1.38).

Speed improvements of the exemplary embodiment of the OFDI procedures and systems over the traditional OCT facilitate the imaging of large tissue volumes at microscopic resolution. Faster image acquisition can also make the imaging less vulnerable to motion artifacts, which can be a desirable trait when dealing with in vivo applications.

### OFDI Imaging in the Pulmonary Airways

To demonstrate the ability of the exemplary OFDI procedures and systems to image the layers of the bronchial mucosa, OFDI imaging may be performed in a swine ex vivo lung. For example, an 18 mm balloon catheter with an optical imaging window of about 5 cm was used to stabilize and centralize the optical inner core with respect to the bronchial mucosa. The exemplary probe was positioned within the left main bronchus extending up into the trachea and traversing the main carina. The balloon was then be inflated, and the inner optical core of the catheter was rotated and translated enabling us to acquire continuous spiral cross-sectional images.

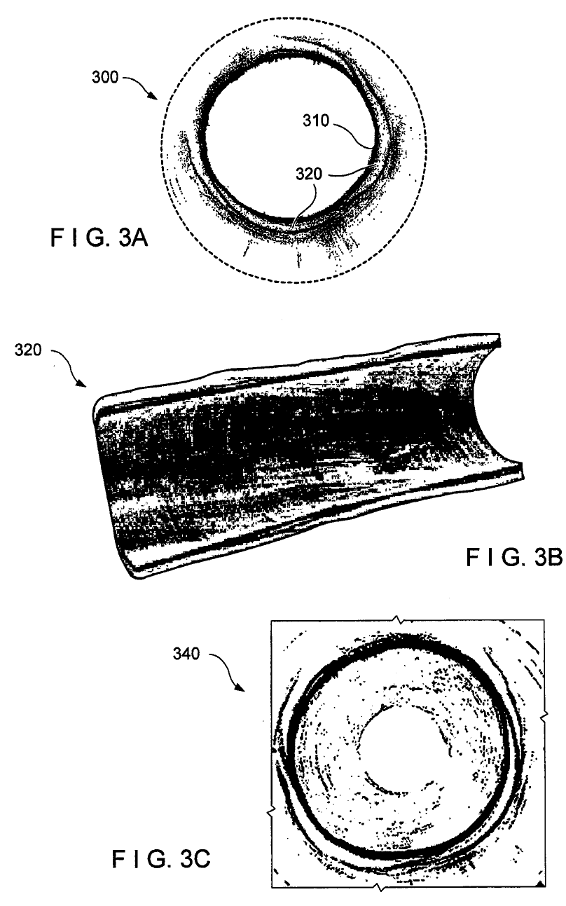

Exemplary comprehensive exemplary volumetric images depicted in FIGS. 3A-3C illustrate an imaging penetration depth of, e.g., approximately 3 mm with an axial resolution of about 8 μm and a transverse and longitudinal pitch of about 20 μm and 50 μm respectively. For example, FIG. 3A shows a cross-section of the exemplary acquired OFDI volumes using the exemplary embodiment of the system according to the present invention. The individual layers of the bronchial airway wall, including the mucosa, the submucosa, the cartilage and the adventitia, are distinguishable. Such exemplary transverse cross-sectional view also illustrates the cartilage layers. The exemplary acquired volume was also subsequently visualized using volume rendering techniques that clearly depict the incomplete cartilage rings of the bronchus, and allows for the three-dimensional appreciation of the bronchial structures. (See, e.g., FIGS. 3B and 3C).

These exemplary results demonstrate that a comprehensive volumetric microscopy in the pulmonary airways using the exemplary OFDI techniques and arrangement is possible, and that the exemplary OFDI imaging facilitates the visualization of the architectural layers in the bronchial wall.

### EXEMPLARY CONCLUSIONS

Thus, with the exemplary OFDI imaging using the exemplary procedures and/or system of biological tissues, is possible to provide a 100-fold increase in imaging speed over traditional OCT. Due to the increased imaging speed, together with certain exemplary optical probe designs, comprehensive microscopy of the pulmonary airways in vivo may be possible. This capability to noninvasively obtain microscopic image data over large epithelial surface areas may aid in early diagnosis and intervention, resulting in a consequent reduction in morbidity and mortality associates with SCC of the lung.

### Exemplary OFDI Catheter for Imaging the Pulmonary Airways In Vivo.

One of the objectives of the present invention is to provide an accurate OFDI-based assessment system and method for the detection and diagnosis of dysplasic changes and early SCC in the bronchial mucosa. Screening the airways for the purpose of detecting possible lesions may prefer, for example, that the catheter function under the control of a standard bronchoscope. Surveillance of identified lesions, or assessment of segments of bronchial mucosa, may prefer the catheter to perform comprehensive volumetric imaging. For example, one exemplary catheter, to facilitate a fluent assessment of the airways without the need to repeatedly change imaging probes, may perform both the screening and surveillance functionalities.

Surveillance To effectively and accurately survey the pulmonary airways, a comprehensive imaging of large areas at microscopic resolution may be desirable, thereby likely eliminating or reducing unnecessary errors that may be attributed to missed diagnoses through sampling error. The exemplary catheter may be configured to acquire an automated circumferential three-dimensional imaging of the airways over predefined bronchial segments. To reduce OFDI imaging time and to facilitate an accurate placement of the catheter, the exemplary probe can serve in an ancillary capacity to the bronchoscope by operating through the access port. The exemplary catheter may also operate independently of the bronchoscope, and can include a stabilization device to centralize and brace the catheter relative to the bronchial wall. This exemplary stabilization device may be permeable to air (or fluid) to facilitate the typically physiological functioning of the airways.

Screening The exemplary catheter retracted into the bronchoscope, with tip still extending, e.g., several millimeters past the distal end of the bronchoscope to facilitate a clear viewing, may operate in the same style as the exemplary catheter described herein above. As the bronchoscope traverses the airways, the exemplary catheter may continuously obtain cross-sectional images of the bronchial wall microstructure. This exemplary catheter can be advantageous over other prior catheters in that, e.g., it may have a more suitable imaging focal length and a stiffer encasing sheath to limit vibrations from the rotating inner core. This exemplary mode of operation can facilitate the physician to perform real time screening of the airway mucosa for the presence of possible pathology.

### Exemplary Pulmonary Airway Catheter Design

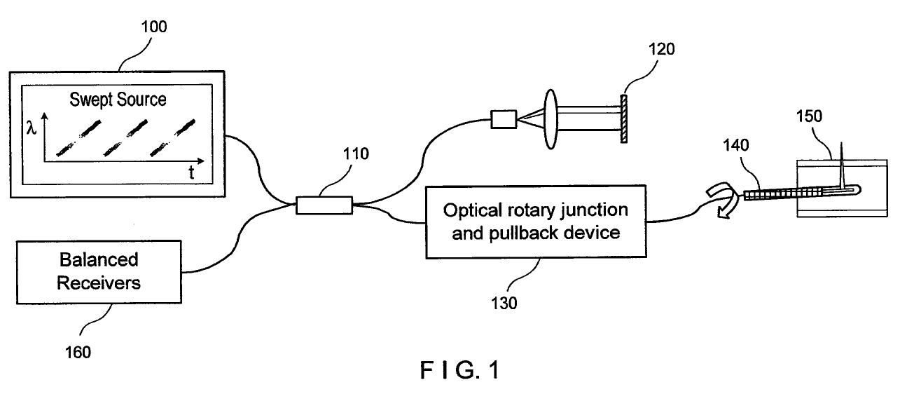

A diagram of an exemplary embodiment of the OFDI apparatus according to the present invention is shown in FIG. 1. This exemplary apparatus can include a wavelength swept source 100, a fiber or free space coupler 110, a reference mirror 120, an OFDI imaging probe 140, an optical rotary junction and pullback device 130 to actuate the probe 140 and a set of balanced receivers 160. Electromagnetic radiation (e.g., light) from the swept source 100 can be used to illuminate both the reference mirror 120 and the tissue sample 150. The spectrally resolved interference signal may be detected by the balanced receivers 160, and the depth profile of the sample 150 may be obtained by determining the Fourier transform. To perform spiral cross-sectional imaging, the OFDI imaging probe 140 can be rotated and translated by the optical rotary junction and pullback device 130.

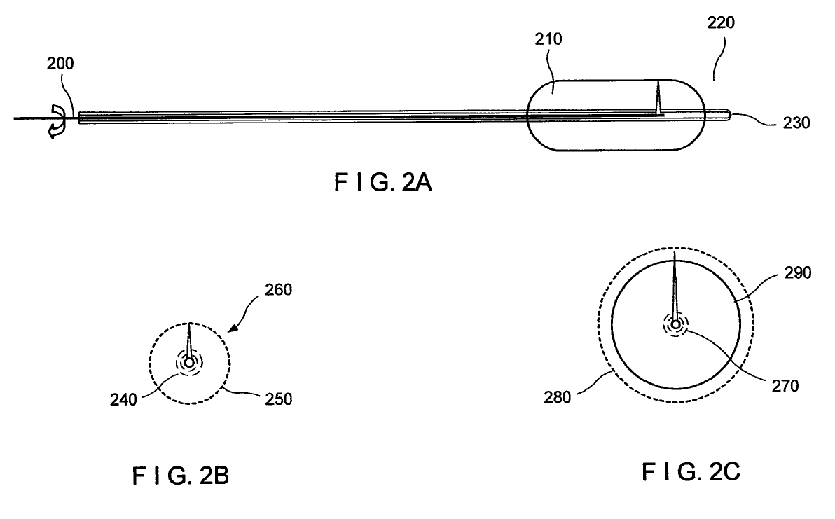

FIG. 2A shows a side view of a diagram of an exemplary embodiment of an OFDI probe configuration according to the present invention. The exemplary OFDI probe configuration can comprise of a single balloon arrangement 210 to center the optical core arrangement 200 within a lumen or hollow organ 220. The optical inner core arrangement 200 may transmit and collect the imaging signal, and can be encased in an outer jacket 230, which can serve to shield a patient from the rotating optical components. The exemplary OFDI probe may acquire helical scans by translating the inner optical core 200 using a pullback device whilst an optical rotary junction simultaneously pivots the core 200. The exemplary OFDI probe configuration may be limited in image ranging depth to, e.g., less than 5 mm. Therefore, as shown in FIG. 2B, in large diameter lumens when the optical core 240 is not centered within the lumen 260, 360 degree imaging may be at least in part lost, as provided in a dashed area 250 in FIG. 2B. As shown in FIG. 2C, centering the optical arrangement 270 within the lumen using the exemplary embodiment comprising of a balloon arrangement 290, can facilitate a 360 degree OFDI imaging of the luminal superficial structure 280.

Preliminary results of three-dimensional imaging of the pulmonary airways obtained from the swine airway ex vivo are shown in FIGS. 3A-3C. The exemplary lumen size of the swine airway was about 18 mm, and therefore it may be important to center the exemplary OFDI optical probe. The exemplary imaged OFDI dataset depicted in FIGS. 3A-3B was obtained using the exemplary embodiment of the OFDI probe described herein with reference to FIGS. 2A-2C. For example, a 360 degree exemplary cross-sectional image 300 is shown in FIG. 3A. The layers of the bronchial mucosa are identifiable as portion(s) 310 including prominent cartilage rings 320. FIGS. 3B and 3C depict exemplary volume renderings 330, 340 of the exemplary three-dimensional OFDI cross-sectional images.

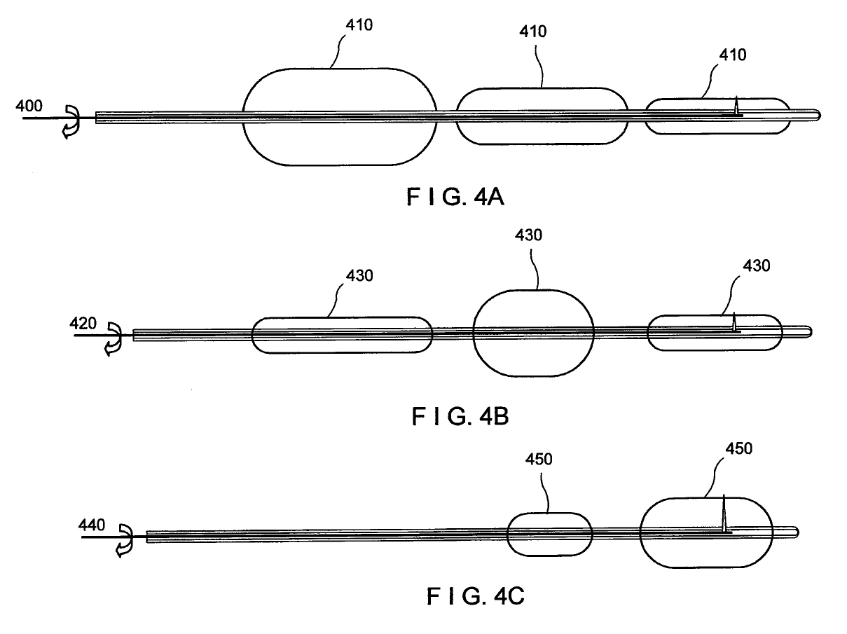

The exemplary luminal diameter of bronchial segments can decrease in the pulmonary airways with an increasing airway generation. Additionally, the lumen diameters may be subject to the presence of strictures or dilated regions within the bronchial tree or other organ to be imaged. One exemplary embodiment of the imaging probe according to the present invention can include a centering arrangement that may accommodate varying luminal diameters, lengths, and topology. FIGS. 4A-4C show side views of exemplary embodiments of the imaging probe comprising a plurality of balloon arrangements in series (e.g., see exemplary balloon arrangement 410, 430, 450 of FIGS. 4A-4C, respectively) to center the respective optical cores 400, 420, 440 with respect to a varying lumen diameter.

In particular, FIG. 4A shows a side view of one exemplary embodiment of the present invention comprising a plurality of balloon arrangements decreasing in diameter 410 to accommodate a lumen diameter decreasing in the distal direction. A side view of another exemplary embodiment of the present invention comprising multiple balloon arrangements with varying diameters 430 to accommodate a dilated luminal diameter is illustrated in FIG. 4B. A side view of a further exemplary embodiment of the present invention is shown in FIG. 4C. The exemplary balloon arrangement 450 of FIG. 4C is designed to accommodate an increasing luminal diameter in the distal direction or a stricture or some other narrowing of the lumen. Various other exemplary balloon arrangements are possible to accommodate for spatially variable lumen diameter, structure, and topology, e.g., in cross-sectional and longitudinal aspects of the specimen.

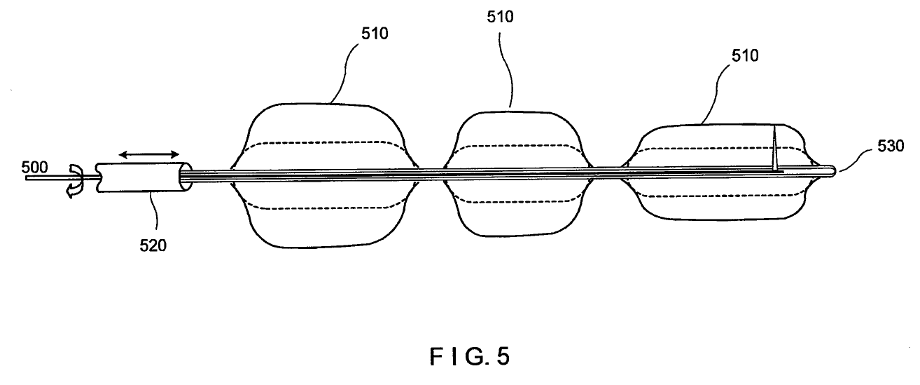

Passage of air, and possibly of fluid, can be important in the normal functioning of the pulmonary airways. Conventional balloon based OFDI centering arrangements can substantially occlude the lumen, and, as a result, may make it difficult to provide for the passage of air or fluid through the airways. FIG. 5 shows a side view of a diagram of an exemplary embodiment of the imaging probe according to the present invention comprising a plurality of wire cage arrangements 510 to center the optical core 500. The exemplary wire cage arrangements 510 can facilitate the passage of at least one of gases or fluids. In one exemplary embodiment, the wire cage arrangements 510 can be attached to an optically transparent sheath or jacket 530 that may encase the optical inner core 500. An exemplary encompassing outer jacket arrangement 520 can activate and/or actuate the wire cage arrangements 510 by sliding over the wire arrangements and determining the number of wire arrangements deployed at any given time. The exemplary wire cage arrangements 510 may be collapsed by retracting the probe into the outer jacket 520. The catheter may then be repositioned and redeployed for imaging additional areas and/or removed entirely from the airway tree.

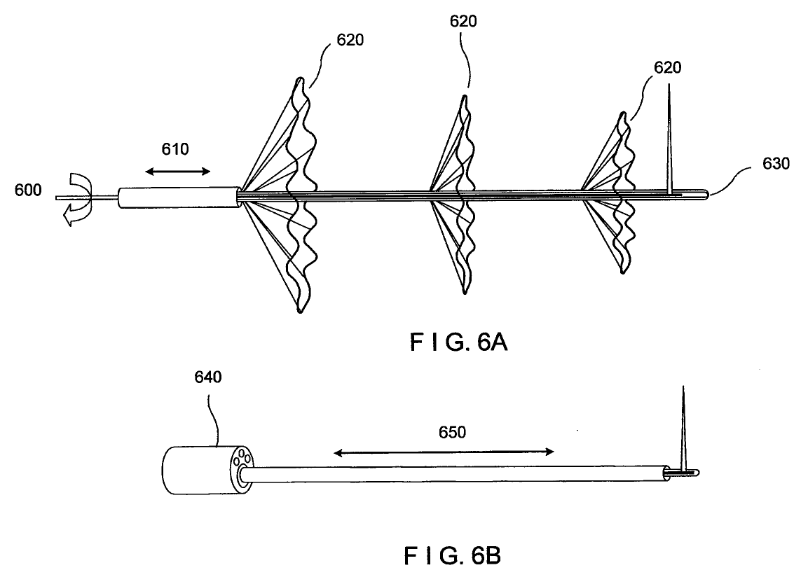

In another exemplary embodiment of the present invention, the imaging probe can comprise at least one or multiple wire or plastic expandable umbrella-like arrangements 620 in series, as shown in an expanded state in FIG. 6A. For example, the umbrella-like arrangements 620 can have variable expansion properties to fit a variety of complex luminal diameters and shapes. The exemplary (e.g., wire or plastic) umbrella arrangements 620 can attach to an optically transparent jacket 630 that may encase an optical imaging core 600 that may be free to rotate and/or translate. The umbrella arrangements 620 can stabilize the catheter with respect to the lumen and to center and the optical imaging core 600. An exemplary encompassing outer jacket arrangement 610 may activate and/or actuate the umbrella arrangements 620 by sliding over the arrangements 620, and determining the number thereof deployed at any given time. FIG. 6B depicts the exemplary embodiment of FIG. 6A in a collapsed state how the umbrella-like arrangements 620 may be collapsed by retracting the exemplary probe into the outer jacket 650. The entire exemplary imaging probe may be passed through the access channel of a standard endoscope or bronchoscope 640 for placement thereof within the bronchial tree, and can be passed through a guide catheter or may be operated in a standalone capacity.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with and/or implement any SEE, OCT system, OFDI system, SD-OCT system or other imaging systems, and for example with those described in International Patent Publication WO2005/047813, U.S. Pat. No. 7,382,949, and U.S. Pat. No. 7,355,716, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for obtaining data for at least one portion within at least one luminal or hollow sample, comprising:
a first optical arrangement configured to transceive at least one electromagnetic radiation to and from the at least one portion;
a second arrangement at least partially enclosing the first arrangement;
at least one third arrangement which is configured to be actuated so as to expand, at least in part, beyond a periphery of the second arrangement, wherein the at least one third arrangement is structured to facilitate at least one of a fluid flow or a gas flow therethrough; and
a fourth arrangement which is structured to at least one of (i) actuate a particular number of the at least one third arrangement or (ii) adjust a distance between at least two outer portions of the at least one third arrangement.

2. The apparatus according to claim 1, wherein the at least one third arrangement is at least one of a wire arrangement or a plastic arrangement.

3. The apparatus according to claim 2, wherein the wire arrangement has at least one wire strand.

4. The apparatus according to claim 2, wherein the wire arrangement is a cage.

5. The apparatus according to claim 1, wherein the at least one third arrangement includes a balloon arrangement.

6. The apparatus according to claim 1, wherein the at least one third arrangement has an approximately circular or elliptical outer periphery, and wherein a circumference of the at least one third arrangement is adjustable by the fourth arrangement.

7. The apparatus according to claim 1, wherein the at least one third arrangement includes a plurality of third arrangements, and wherein the fourth arrangement actuates the particular number of the third arrangements.

8. The apparatus according to claim 7, wherein the third arrangements are spaced apart from one another by at least one predetermined distance, and wherein the predetermined distance is provided such that upon a completed collapse of each of the third arrangements, outer portions of the each of the third arrangements are prevented from substantially overlapping one another.

9. The apparatus according to claim 1, wherein the at least one third arrangement is statically connected to the second arrangement, and the at least one third arrangement translates over at least one portion of the at least one third arrangement.

10. The apparatus according to claim 1, wherein the at least one third arrangement adjusts the distance by translating at least one of the at least one third arrangement or the fourth arrangement with respect to one another.

11. The apparatus according to claim 1, wherein, in at least a partially expanded state, the at least one third arrangement has an approximate shape of a cone.

12. The apparatus according to claim 1, wherein the at least one portion is within an airway of a patient, and wherein the at least one third arrangement is structured to be insertable into the airway.

13. The apparatus according to claim 1, wherein the distance is a radius of an outer periphery of the at least one third arrangement.

14. The apparatus according to claim 1, further comprising a fifth arrangement that substantially surrounds the fourth arrangement.

15. The apparatus according to claim 14, wherein the fifth arrangement is at least one of an endoscope, a laparascope, a bronchoscope, a cystoscope or a guide catheter.

16. An apparatus for obtaining data from or treating at least one portion within at least one luminal or hollow sample, comprising:
a first arrangement configured to transmit at least one electromagnetic radiation to and from the at least one portion;
a second arrangement at least partially enclosing the first arrangement; and
a plurality of third arrangements which are configured to be actuated so as to expand, at least in part, beyond a periphery of the second arrangement.

17. The apparatus according to claim 16, wherein at least one of the third arrangements is structured to facilitate at least one of a fluid flow or a gas flow therethrough.

18. The apparatus according to claim 16, further comprising a fourth arrangement which is structured to at least one of (i) actuate a particular number of the third arrangements or (ii) adjust a distance between at least two outer portions of at least one of the third arrangements.

19. The apparatus according to claim 18, wherein the third arrangements are spaced apart from one another by at least one predetermined distance, and wherein the predetermined distance is provided such that upon a completed collapse of each of the third arrangements, outer portions of the each of the third arrangements are prevented from substantially overlapping one another.

20. The apparatus according to claim 16, wherein the third arrangements are configured to be actuated to expand so as to be associated with a plurality of portions within the at least one luminal or hollow sample.
